(19) United States
(12) Patent Application Publication
(10) Pub. No.: US 2024/0024617 A1
(43) Pub. Date: Jan. 25, 2024

(54) SLEEP IMPROVEMENT DEVICE AND SLEEP IMPROVEMENT METHOD

(71) Applicant: SLEEP SYSTEM LABORATORY INC., Tokyo (JP)

(72) Inventor: Shin NEMOTO, Chiba (JP)

(21) Appl. No.: 18/025,925

(22) PCT Filed: Jun. 11, 2021
(86) PCT No.: PCT/JP2021/024264

§ 371 Date: Mar. 13, 2023

(30) Foreign Application Priority Data
Sep. 25, 2020 (JP) 2020-161474

(51) Int. Cl. A61M 21/02; A61B 5/00; G16H 20/00; G16H 50/50

(52) U.S. Cl. CPC A61M 21/02; A61B 5/4812; G16H 20/00; G16H 50/50; A61M 2230/06

(57) ABSTRACT

[Problem]

The sleep improvement device provides a sleep improvement plan of lifestyle improvement suited to an individual in order to improve the sleep state with a simple and low-cost configuration.

[Method of Settlement]

The sleep evaluation index for model construction calculated based on the time of each sleep stage of the user, (2) the energy value of at least the extremely low frequency component before bedtime calculated by the autonomic calculation section 12, (3) the sleep evaluation prediction model for constructing a sleep evaluation prediction model equation specific to the user. (4) a sleep improvement proposal proposal section 7, which proposes a sleep improvement proposal through lifestyle improvement based on the constructed sleep evaluation prediction model formula and the energy value of the extremely low frequency component.

### TECHNICAL AREA

This invention relates to a sleep improvement device and a sleep improvement method that contribute to improving sleep conditions by providing a sleep improvement plan through lifestyle improvement suited to the user to improve sleep conditions.

### BACKGROUND TECHNOLOGY

Sleep is said to be a barometer of health, and many people experience in their daily lives that if they have a good night's sleep and wake up feeling good, they will feel refreshed and healthy upon awakening. On the other hand, when a person has insomnia or insomnia tendency, or when a person is forced to sleep with his/her day and night life reversed due to late-night work, etc., the mood after waking up is often not good. In other words, whether consciously or unconsciously, the state of sleep influences mood and behavior upon subsequent awakening, which in turn determines the quality of daytime activities after awakening. Thus, sleep is a factor that has an important influence on human physical and mental activity, and a good night's sleep is a guarantee of physically and mentally healthy daily activities.

Sleep is said to be a reflection of daytime living conditions, but it has been considered difficult to predict sleep evaluation because physical fatigue and psychological tension have different characteristics depending on the individual. Therefore, the main treatment by physicians for insomnia is the administration of sleep inducing agents, and technologies related to various sleep inducing agents have been proposed, for example, as described in Patent Document 1. In addition, attempts have been made to control the environmental temperature during sleep for the purpose of ensuring comfortable sleep. As technologies embodying such attempts, as described in Patent Literature 2 to 4, for example, those that determine sleep depth and sleep state based on biological signals and control the sleep environment temperature according to the determined sleep depth and sleep state have been proposed.

### PRIOR TECHNICAL DOCUMENTS

### Patent Document

[patent document 1] Japanese Patent Laid-Open No. 2018-044006 bulletin
[patent document 2] Japanese Patent Laid-Open No. 2008-119454 bulletin
[patent document 3] Japanese Patent Laid-Open No. 2009-247846 bulletin
[patent document 4] Japanese Patent Laid-Open No. 2006-198023 bulletin

### SUMMARY OF THE INVENTION

### Problem to be Solved by this Invention

However, long-term administration of sleep inducing drugs is problematic because of their adverse effects, such as promotion of dementia. Therefore, it is desirable to eliminate insomnia by improving one's lifestyle. As with lifestyle-related diseases, we believe that the correct approach is to solve this problem by increasing physical activity and improving physical functions.

The purpose of this invention is to provide a sleep improvement device and a sleep improvement method that can provide a personalized index for improving sleep conditions and contribute to improving sleep conditions based on this index, under a simple and low-cost configuration.

### Means for Solving the Problem

The sleep improvement device for the present invention, which achieves the above-mentioned purposes, is a sleep improvement device that contributes to improving the sleep state by providing a sleep improvement plan through lifestyle improvement suited to the user to improve the sleep state, comprising:

(1) a heart rate signal measuring means for measuring the user's heart rate signal, and

(2) autonomic nerve calculation means for calculating an energy value of an extremely low frequency component (VLF) of at least 0.003 to 0.04 Hz among the autonomic nerve components of the user based on the heart rate signal measured by the heart rate signal measuring means,

(3) biometric signal detection means for detecting a biometric signal of the user, and based on the biometric signals detected by the biometric signal detecting means

(4) a means for determining each sleep stage of a user during sleep and determining the time of each sleep stage, a means for calculating a sleep evaluation index for model building based on the time of each sleep stage determined by the means for determining sleep stage, a means for constructing a sleep evaluation prediction model equation specific to a user based on the sleep evaluation index for model building calculated by the means for calculating sleep evaluation index, and a means for calculating an autonomic component calculated by the means for calculating autonomic component.

(5) a means for constructing a sleep evaluation prediction model equation specific to a user based on the sleep evaluation index for model construction calculated by the sleep evaluation index calculation means and the energy value of at least the very low frequency component (VLF) before bedtime calculated by the autonomic component calculation means, and

(6) Calculating a predictive sleep evaluation index based on the energy value of at least the very low frequency component (VLF) calculated by the autonomic calculation means afterward, and proposing a sleep improvement plan by lifestyle improvement to make the energy value of at least the very low frequency component (VLF) exceed a certain value, based on the calculated predictive sleep evaluation index. The sleep improvement plan proposal method is characterized in that it proposes a sleep improvement plan based on the calculated predictive sleep evaluation index.

The method of improving sleep, which achieves the above-mentioned purposes, is a method of improving sleep by providing a sleep improvement plan through lifestyle improvement suited to the user to improve the sleep condition, comprising:

(1) a heart rate signal measuring process for measuring a heart rate signal of the user by a predetermined heart rate signal measuring means, and (2) a signal processing processor for processing the signal. an autonomic nerve calculation process in which a processor performing signal processing calculates an energy value of an extremely low frequency component (VLF) of at least 0.003 to 0.04 Hz among the autonomic nerve components of the user based on the heartbeat signal measured in the heartbeat signal measurement process, and a biological signal detection process in which a biological signal of the user is detected by the predetermined biological signal detection means.

(2) a biometric signal detecting process for detecting the biometric signal of the user by the predetermined biometric signal detecting means, the processor determining each sleep stage of the user during sleep based on the biometric signal detected in the biometric signal detecting process, and determining the time of each sleep stage, and

(3) The processor calculates a sleep evaluation index for model building based on the time of each sleep stage determined in the sleep stage determination process, and

(4) the processor constructs a user-specific sleep evaluation prediction model equation based on the sleep evaluation index for model building calculated in the sleep evaluation index calculation process and (5) the energy value of at least the very low frequency component (VLF) before bedtime calculated in the autonomic nerve component calculation process.

(6) the processor calculates a predictive sleep evaluation index based on the sleep evaluation prediction model equation constructed in the sleep evaluation prediction model equation construction process and the energy value of at least an extremely low frequency component (VLF) subsequently calculated in the autonomic calculation process, and based on the calculated predictive sleep evaluation index The sleep improvement proposal process is characterized in that it proposes a sleep improvement plan by improving the lifestyle so that the energy value of at least the extremely low frequency component (VLF) exceeds a certain value.

The sleep improvement device and sleep improvement method of the present invention constructs a user-specific sleep evaluation prediction model equation based on a sleep evaluation index for model building based on the time of sleep stage and the extremely low frequency component (VLF) of the autonomic component, and based on this sleep evaluation prediction model equation, predicts the then The sleep evaluation index is calculated, and based on the calculated predicted sleep evaluation index, a sleep improvement plan is proposed through lifestyle improvement that will at least raise the energy value of the very low frequency component (VLF) above a certain value.

### Effect of the Invention

In this invention, it is possible to provide a sleep improvement plan by lifestyle improvement suited to the individual to improve sleep conditions under a simple and low-cost configuration, and based on this sleep improvement plan, it is possible to contribute to the improvement of sleep conditions.

### BRIEF DESCRIPTION OF THE FIGURES

FIG. 1 This figure shows the configuration of the sleep improvement device shown as an embodiment of the present invention.

FIG. 2 shows the configuration of the sleep improvement device shown as an embodiment of the present invention, and is a partial cross-sectional view when viewed from the arrow-viewing direction in FIG. 1.

FIG. 3 This figure shows an example of a comparison between the measurement results of the autonomic nervous system component obtained from the heartbeat signal before bedtime and the sleep evaluation index after bedtime.

FIG. 4 shows the cross-correlation coefficient between the autonomic component and the sleep evaluation index in the example shown in FIG. 4.

FIG. 5 This figure shows the configuration of the other biometric signal detection unit.

### FORM TO CARRY OUT INVENTION

The following is a detailed description of specific embodiments in which the invention is applied, with reference to the drawings.

This form of sleep improvement device is based on the user's bio-signals and provides a sleep improvement plan through lifestyle improvement suited to the individual user to improve his/her sleep state.

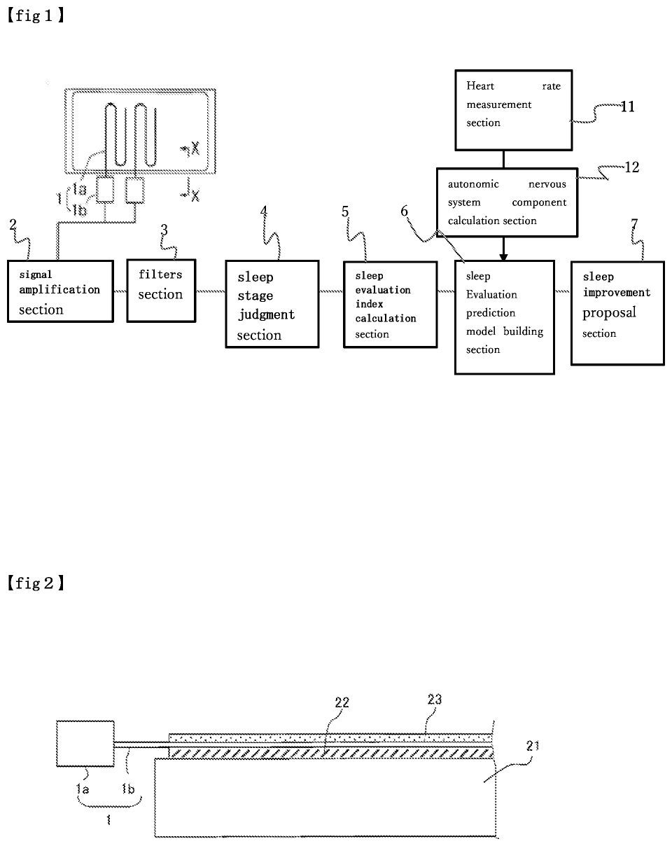

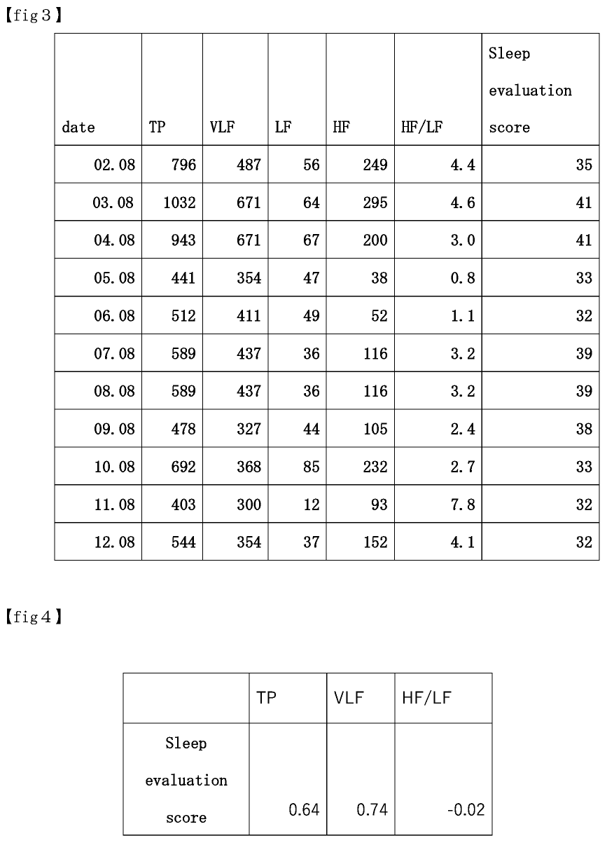

(1) FIG. 1 shows the configuration of the sleep improvement device shown as an embodiment of the invention, representing the processing of the device as a block, and FIG. 2 shows a partial cross-sectional view of the device when viewed from the arrow-view direction in FIG. 1.

(2) The sleep improvement device consists of a biometric signal detection unit 1 that detects biometric signals of a user lying on a bed 21, a signal amplification unit 2 that amplifies the biometric signals detected by the biometric signal detection unit 1,

(3) a filter unit 3 that applies filtering processing to the biometric signals amplified by the signal amplification unit 2, and a sleep monitoring unit 4 that performs sleep monitoring based on the biometric signals passing through the filter unit 3. a sleep stage determination unit 5 that determines the sleep stage based on the biometric signal that has passed through the filter unit 3, and

(4) Based on the time of each sleep stage determined by this sleep stage determination section 4. Sleep evaluation index calculation section 5, which calculates a sleep evaluation index for model construction as described below, (5) sleep evaluation prediction model construction section 6, which constructs a sleep evaluation prediction model equation specific to the user based on the sleep evaluation index for model construction calculated by this sleep evaluation index calculation section, and (6) sleep evaluation prediction model construction section 7, which proposes a sleep improvement plan based on the sleep evaluation prediction model equation constructed by this sleep evaluation prediction model construction section 6.

(7) The sleep improvement device also has a heart rate measurement unit 11 that measures the user's heart rate signal and a

(8) The autonomic nervous system component calculation section 12 calculates the autonomic nervous system component based on the heart rate signal measured by the heart rate measurement section 11.

(9) Of these sections, at least the sleep stage determination section 4, sleep evaluation index calculation section 5, sleep evaluation prediction model equation construction section 6, sleep improvement suggestion section 7, and autonomic component measurement section 12 can be implemented as programs that can be executed using hardware such as a central processing unit (CPU) or memory in a signal processing computer. The 5, 6, 7, and 12 are implemented as programs that can be executed using hardware such as a CPU (Central Processing Unit) or memory in a computer that performs signal processing. They can also be implemented using a dedicated processor such as a DSP (Digital Processing Unit) mounted on an expansion board that can be attached to the computer.

(10) The sleep improvement device can also be implemented by configuring the biometric signal detection unit 1, signal amplification unit 2, and filter unit 3 as a single sleeper-type device, and by transmitting the measured data from this sleeper-type device to a cloud server, the sleep stage judgment unit 4, sleep evaluation index calculation unit 5, sleep evaluation prediction The cloud server may perform the processes of the sleep stage determination section 4, sleep evaluation index calculation section 5, sleep evaluation prediction section 6, and sleep improvement proposal section 7.

The biometric signal detection part 1 is a noninvasive and nonrestrictive sensor that detects minute biometric signals of the user. Specifically, the biometric signal detection unit 1 consists of a pressure detection tube 1a and a differential pressure sensor 1b, which is a sensor that detects minute pressure fluctuations in the air contained within the pressure detection tube 1a, and constitutes a non-invasive and non-binding means of detecting biometric signals.

As the pressure sensing tube 1a, it should have adequate elasticity so that the internal pressure fluctuates in response to the pressure variation range of the biological signal. As the pressure sensing tube 1a, the hollow volume of the tube should be appropriately selected to transmit pressure changes to the differential pressure sensor 1b at an appropriate response speed. If the pressure sensing tube 1a cannot satisfy both moderate elasticity and hollow volume at the same time, the hollow section of the pressure sensing tube 1a can be loaded with a core wire of appropriate thickness over the entire length of the tube to take up the appropriate volume of the hollow section.

The pressure sensing tube 1a is placed on a hard sheet 22 laid on a bed 21. In the sleep stage evaluation device, a cushion sheet 23 having elasticity is laid on the hard sheet 22, and the user lies on the pressure detection tube 1a. The pressure detection tube 1a may be configured to be incorporated into the cushion sheet 23 or the like, thereby stabilizing the position of the pressure detection tube 1a.

(1) The differential pressure sensor 1b is a sensor that detects minute pressure fluctuations. In this embodiment, a condenser microphone type for low frequency is used as the microdifferential pressure sensor 1b, but it is not limited to this type, as long as it has an appropriate resolution and dynamic range.

(2) The low-frequency condenser microphone used in this embodiment has greatly improved the characteristics of the low-frequency range by providing a chamber behind the pressure-sensing surface, in contrast to ordinary acoustic microphones, which are not designed for the low-frequency range. It is suitable for detecting minute pressure fluctuations in the pressure sensing tube 1a.

(3) The condenser microphone is also excellent for measuring minute differential pressure, with a resolution of 0.2 Pa and a dynamic range of about 50 Pa, which is several times higher than that of commonly used ceramic-based minute differential pressure sensors, and is suitable for detecting minute pressure applied by biological signals through the body surface to the pressure sensing tube It is suitable for detecting minute pressure applied to 1A and can detect minute body movements with high sensitivity.

In this embodiment, two sets of pressure sensing tubes 1a are provided, so that one set detects biological signals in the chest area of the user and the other set detects signals in the buttocks area of the user. The device is configured to detect biological signals regardless of the sleeping posture of the user.

In the sleep improvement device, the pressure detection tubes 1a may be configured to be placed only at one of the chest or buttocks area. The biological signals detected by the biological signal detection unit 1 are supplied to the signal amplification unit 2. This non-invasive and non-restrictive configuration for detecting biological signals allows the sleep improvement device to be easily used in daily life and is extremely suitable for use by the elderly in particular.

I

The signal amplification section 2 amplifies the signals detected by the biometric signal detection section 1 so that they can be processed in subsequent processing steps, and also performs appropriate signal shaping processing, such as removing apparently abnormal level signals. The biological signals amplified by the signal amplification section 2 are supplied to the filter section 3.

(1) The filter section 3 extracts the heartbeat signal by removing unnecessary signals from the biological signal amplified by the signal amplifier section 2 using a bandpass filter or the like.

(2) In other words, the biological signal detected by the biological signal detection unit 1 is a mixed signal of various vibrations emitted from the human body, and includes various signals such as body motion signals due to turning over, etc., in addition to the heartbeat signal. Among these, the heartbeat signal is a biological signal in which pressure changes (i.e., blood pressure) based on the pumping function of the heart become vibrations.

(3) In the sleep improvement device, the heartbeat signal is recognized as a heartbeat signal by extracting it with the filter section 3. The heartbeat signal that has passed through the filter section 3 is supplied to the sleep stage determination section 4. The sample period of the heartbeat signal is 4 milliseconds.

The sleep stage determination section 4 determines the sleep stage (wakefulness, REM sleep, shallow sleep, and deep sleep) according to the international sleep depth determination criteria by the so-called polysomnograph (PSG).

Specifically, the sleep stage determination section 4 employs the methods described in, for example, JP-A2016-022276, JP-A2016-202463, JP-A2018-029772, etc. by the applicant to determine the sleeping The user's sleep stage, i.e., awake stage, REM sleep stage, shallow sleep stage, and deep sleep stage, is determined by determining the type of sleep stage of the user.

The sleep stage determination section 4 determines the time of each determined sleep stage and supplies the information on the time to the sleep evaluation index calculation section 5.

The sleep evaluation index calculation section 5 calculates a sleep evaluation index for model construction based on the time information for each sleep stage determined by the sleep stage determination section 4 in order to construct a user-specific sleep evaluation prediction model equation, which is described later.

The sleep evaluation index calculation section 5 supplies the calculated sleep evaluation index for model construction to the sleep evaluation prediction model equation construction section 6.

The sleep evaluation prediction model equation construction section 6 constructs a user-specific sleep evaluation prediction model equation based on the energy values of the autonomic components calculated by the autonomic component calculation section 12, especially the very low frequency component (VLF) of 0.003 to 0.04 Hz. The details of this are described below.

(1) The sleep improvement proposal section 7 outputs a sleep improvement proposal based on the energy value of the autonomic component calculated by the autonomic component calculation section 12, especially the very low frequency component (VLF) of 0.003 to 0.04 Hz, and the energy value of the autonomic component calculated by the autonomic component calculation section 12, especially the very low frequency component of 0.003 to 0.04 Hz.

(2) Based on the sleep evaluation prediction model equation constructed by the sleep evaluation prediction model equation construction section 6, the sleep improvement plan by lifestyle improvement suited to the user to improve the sleep state is output.

(3) Specifically, the Sleep Improvement Proposal 7 proposes a sleep improvement plan that enables a good sleep by improving the daytime lifestyle so that the energy value of the very low frequency component (VLF) is above a certain value.

(4) The energy value of the very low frequency component (VLF) is extremely lowered when the user becomes emotional or stressed.

(5) Therefore, based on the user's daily life recorded using the so-called VAS method, etc., including both psychological and physical aspects, and taking into consideration the phenomena that affect the extremely low frequency component (VLF), we propose a lifestyle that would make the energy value of the extremely low frequency component (VLF) exceed a certain value as a sleep improvement plan.

(6) The sleep improvement proposal proposal section 7 outputs the sleep improvement proposal and displays it on a display device not shown in the figure, prints it by a printing device, or stores it as data in a memory device.

The heart rate measurement section 11 measures the heart rate during the daytime (when the user is awake), especially before entering the bed, and measures the user's heart rate signal (pulse wave) using, for example, an existing heart rate measuring device that inserts a sensor at the fingertip or a wearable type sensor such as a wristwatch type.

The heart rate measurement unit 11 supplies the measured heart rate signal to the autonomic nerve component calculation unit 12.

(1) The autonomic component calculation unit 12 calculates the autonomic component based on the heart rate signal measured by the heart rate measurement unit 11.

(2) The heart rate measurement unit 11 and the autonomic component calculation unit 12 are usually configured as separate devices from the bedside type device, and from the viewpoint of convenience of handling, it is especially desirable to use a wearable type sensor.

(3) Such a wearable type sensor can transmit the heartbeat signal or the information on the autonomic component obtained therefrom to the outside through wireless communication such as Bluetooth (registered trademark), for example.

(4) More specifically, the wearable type sensor can transmit the heart rate signal or the information on the autonomic component obtained therefrom via wireless communication to a cloud server, etc., which constitutes the sleep evaluation prediction model formula construction part 6 and the sleep improvement suggestion part 7.

(5) The wearable type sensor may also transmit the heart rate signal or the information on the autonomic component obtained from it to a portable terminal carried by the user, and from this portable terminal to a cloud server or other device.

(6) The autonomic component calculation unit 12 can calculate the sympathetic component (LF), the parasympathetic component (HF), the energy value of the very low frequency component (VLF) of 0.003 to 0.04 Hz, and the autonomic component total power (TP), which is the sum of these components, as the autonomic component.

(7) The autonomic component calculated by the autonomic component calculation section 12 is supplied to the sleep evaluation prediction model construction section 6 and the sleep improvement suggestion section 7.

Such a sleep improvement device calculates a sleep evaluation index based on the following principles

The sleep improvement device constructs a user-specific sleep evaluation prediction model equation based on the user's daytime lifestyle and actual data on sleep evaluation indicators.

The sleep improvement device then proposes a sleep improvement plan that will improve the user's daytime lifestyle, thereby enabling the user to sleep better.

(Current sleep evaluation is based on the proportion of time spent in sleep stages (wakefulness, REM sleep, shallow sleep, and deep sleep) according to the international sleep depth criteria by the so-called polysomnograph (PSG).

(2) Specifically, when the weight coefficients α=20, β=8, and γ=1, the sleep evaluation index is calculated as follows: Sleep evaluation index=20×(deep sleep time)+6×(shallow sleep time)+(REM sleep time).

(3) improvement device detects biological signals in a non-invasive and unrestrained manner to determine the time of each sleep stage by the sleep stage determination unit 4, and the sleep evaluation index for model building is calculated by the sleep evaluation index calculation unit 5.

(1) Next, in the sleep improvement device, the sleep evaluation prediction model construction section 6 constructs a user-specific sleep evaluation prediction model equation using the user's quantified daytime activity level.

(2) Specifically, as described above, the sleep evaluation prediction model equation construction section 6 uses the autonomic component quantified by the autonomic component calculation section 12. This quantification of the autonomic component is based on the following hypothesis.

(1) It can be inferred that the sleep evaluation index has a proportional relationship with the degree of physical and mental fatigue during the daytime and the sleep status of the previous day.

(2) It can also be inferred that there is a relationship between the sleep evaluation index and the user's VAS (daily activity record), a sensory index of sleep goodness or badness.

(3) In particular, when the amount of sustained activity is high, the energy values of the above-mentioned autonomic component total power (TP) and very low frequency component (VLF) (0.003 to 0.04 Hz, 5 minutes to 25 seconds) become high, and the sleep evaluation index becomes high, which indicates that the user can sleep well.

(4) The extremely low frequency component (VLF) may be an indicator of improvement by finding the exercise content that suits the individual user, since the amount of sustained activity varies depending on the content and method of exercise.

(1) It is also known that when autonomic nervous system disorders occur, the autonomic balance is skewed toward sympathetic dominance, which promotes cardiovascular degeneration, especially in the elderly.

(2) Decreased heart rate variability may be due to increased sympathetic tone and decreased parasympathetic tone. This is associated with mortality from heart failure, coronary artery disease, and acute myocardial infarction.

(3) Total heart rate variability can be evaluated by determining the autonomic component through power spectrum analysis of the frequency component of the periodic variability of the heartbeat.

(4) Autonomic nervous system disorders are mainly ameliorated by exercise. Diabetes mellitus, for example, is a typical example of vascular effects.

(5) These autonomic nerves also affect the state of sleep. When autonomic nerve disorders occur, the balance of the autonomic nerves becomes skewed toward sympathetic dominance, the tension component increases, and sleep becomes difficult. This is a common phenomenon seen in the elderly.

(1) Furthermore, the very low frequency component (VLF) reflects vasomotor activity, the renin-angiotensin system (hormonal system), and thermoregulation.

(2) Here, in deep sleep, the body temperature decreases. If the energy value of the very low frequency component (VLF) is low, it can be inferred that thermoregulation does not function and causes poor sleep.

(3) The middle frequency component (0.05-0.20 Hz) of the sympathetic component (LF) is a tension component reflecting the baroreceptor system.

(4) The high-frequency component (0.20-0.35 Hz) of the parasympathetic component (HF) is the relaxation component that reflects respiratory variability.

(5) Therefore, the more the parasympathetic component (HF) is dominant, i.e., the higher the parasympathetic component (HF)/sympathetic component (LF), the more the body relaxes and sleeps better in general.

(6) Furthermore, the autonomic component total power (TP) is the energy value of the very low frequency component (VLF)+sympathetic component (LF)+parasympathetic component (HF), and the larger this value is, the greater the stress tolerance.

(7) Therefore, the larger the autonomic component total power (TP), the better the sleep.

Therefore, good sleep is when the energy value of the very low frequency component (VLF) and the total power of the autonomic component (TP) are large before bedtime. It can be said that finding exercise and lifestyle activities that increase these values above a certain level in daily life will lead to improved sleep.

(1) To verify these hypotheses, FIG. 3 shows an example of comparing the measurement results of the autonomic component obtained from the heartbeat signal before bedtime and the sleep evaluation index after bedtime for one subject.

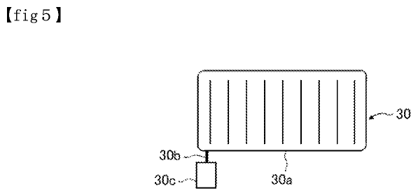

(2) The measurement was performed by the sleep improvement device shown in FIG. 1, and the cross-correlation coefficient between this autonomic component and the sleep evaluation index was calculated as shown in FIG. 4.

(3) Among the autonomic nerve components, it can be seen that the energy value of the very low frequency component (VLF) has a particularly high correlation with the sleep evaluation index.

(4) From this correlation, when constructing a sleep evaluation prediction model formula for the subject, y=0.0204x+27.2.

(5) Here, y is the sleep evaluation index and x is the energy value (mS2) of the very low frequency component (VLF) before bedtime. Needless to say, this sleep evaluation prediction model equation varies from user to user.

In the sleep improvement device, the sleep evaluation prediction model equation is constructed based on such verification results to derive a sleep evaluation index for model construction. Based on the obtained sleep evaluation index for model construction, the sleep evaluation prediction model equation is constructed.

(1) Specifically, in the sleep improvement device, the process of calculating the autonomic component before the user's bedtime is performed by the autonomic component calculation unit 12.

(2) For example, this is done over a period of 10 days. At this time, what kind of daily life, such as the content of exercise, is recorded separately when the energy value of the very-low-frequency component (VLF) is high.

(3) The content of exercise, for example, may vary from person to person, but exercises that stimulate blood vessels by stretching, running, etc. may be included.

(4) In the sleep improvement device, the sleep evaluation index calculation section 5 also calculates a sleep evaluation index for model building based on the time spent in each sleep stage. The same process is performed over a period of 10 days, for example.

(5) The sleep improvement device then constructs a sleep evaluation prediction model equation by the sleep evaluation prediction model construction section 6 based on the obtained data.

(1) In order to make the sleep evaluation prediction model equation an ideal prediction model equation that does not depend on the user's physical defects or environmental conditions such as the bedroom, the following phenomena should not be measured for the construction of the prediction model equation on days when they occur. (2) That is, in a sleep improvement device,

(2) 1) when a physical defect occurs, such as sudden leg cramps or abdominal pain during the night,

(3)2) when the sleep environment deteriorates, such as when the bedding is inappropriate for the user and the body gets cold or when the user cannot sleep due to surrounding noise, etc., and

(4) 3) When the user is slumbering and staying in bed even though he/she is not sleepy,

(5)4) When the user is emotionally elated after the measurement of the energy value of the extremely low frequency component (VLF), as in the case of an argument with another person (in this case, the extremely low frequency component (VLF) is extremely low), it is desirable to exclude the user from the measurement target.

(1) In the sleep improvement device, once the sleep evaluation prediction model equation is constructed in this way, the autonomic component calculation section 12 is used to measure and obtain the energy value of the very low frequency component (VLF) twice, for example, in the evening and before bedtime, and the sleep improvement suggestion section 7 calculates the predicted sleep evaluation index at that time by fitting the energy value of the very low frequency component (VLF) to the sleep evaluation prediction model equation. The predicted sleep evaluation index is calculated by fitting the energy value of the extremely low frequency component (VLF) to the sleep evaluation prediction model formula by the sleep improvement proposal section 7.

(2) The sleep improvement device then proposes a sleep improvement plan by the sleep improvement plan proposal section 7 based on the calculated predictive sleep evaluation index.

(3) For example, the sleep improvement suggestion section 7 knows in advance a sleep evaluation index that is above a certain value at which the user sleeps well, and then proposes a sleep improvement suggestion based on the predictive sleep evaluation model equation.

If the predicted sleep evaluation index based on the energy value of the very low frequency component (VLF) calculated from the sleep evaluation prediction model equation is more than a certain value different from the previously known sleep evaluation index, the sleep improvement suggestion section 7 makes a recommendation for prescribed exercise, bathing, meditation for psychological improvement, etc. that would increase the energy value of the very low frequency component (VLF) above a certain value. Recommendations are made to increase the energy value of this very low frequency component (VLF) above a certain value.

(4) Based on this proposal, if the energy value of the very low frequency component (VLF) is measured in the evening, the user can improve the VLF by about 10-30% by exercising, bathing, or meditating for psychological improvement before bed that day, which is a considerable improvement. This may lead to a good sleep on that day.

(5) Furthermore, reflecting on the day's living conditions, one can improve sleep by exercising or meditating for psychological improvement on the next day, etc.

(6) After constructing the sleep evaluation prediction model equation, it is no longer necessary to calculate the sleep evaluation index for model construction by the sleep evaluation index calculation section 5, but it is desirable to record it daily and use it as reference information for comparison with the predicted sleep index evaluation by the sleep improvement proposal section 7, etc.

(1) By performing this process, the sleep improvement device enables the user to grasp whether sleep is good or bad in the evening and before going to bed, and encourages the user to improve sleep and to reflect on the day's lifestyle.

(2) In particular, the user can improve his/her sleep condition by finding ways to increase the energy value of the very low frequency component (VLF) and by finding ways to improve his/her living conditions, including psychological aspects.

(3) This allows users to set their own target values for reevaluating their living conditions (psychological and physical) and motivates them to improve their living conditions. This method can also be used as an indicator for exercise therapy in the rehabilitation of lifestyle-related diseases such as diabetes.

The invention is not limited to the forms described above.

(1) For example, instead of using the hollow tube described above, an air-mat type detection method as shown in FIG. 6 may be used for the biometric signal detection part 1.

(2) That is, the biometric signal detection unit 30 shown in FIG. 6 is composed of an air tube 30b connected to one end of an air mat 30a with air filled inside, and furthermore, a differential pressure sensor 30c is connected to this air tube 30b.

(3) The differential pressure sensor 30c can be the same as that described in the case of the biometric signal detection unit 1 using a hollow tube.

(1) Although we have described a configuration with a bed-type device, independent heart rate measurement unit 11 and autonomic calculation unit 12, and a cloud server, the following configuration is not limited to this type of device configuration.

(2) However, the present invention is not limited to this type of device configuration, but can take various forms as long as the configuration allows each part to perform its function.

Thus, it goes without saying that the present invention can be modified as needed without departing from its intent.

### EXPLANATION OF THE MARK

1, 30 Biological Signal Detection Section
1a Pressure detection tube
1b, 30c Differential pressure sensor
2 Signal Amplification Section
3 Filter section
4 Sleep Stage Judgment Section
5 Sleep Evaluation Index Calculation Section
6 Sleep Evaluation Prediction Model Building Section
7 Sleep Improvement Proposal Proposal
11 Heart Rate Measurement Section
12 Autonomic Nervous System Component Calculation Section
21 Sleeping table
22 Rigid sheet
23 Cushion sheet
30a Air mat
30b Air tube

1. A sleep improvement device that contributes to the improvement of sleep conditions by providing sleep improvement plans based on lifestyle improvements that suit the user to improve sleep conditions,
And a heart rate signal measuring means for measuring the heart rate signal of said user And autonomic nerve calculation means for calculating the energy value of an extremely low frequency component (VLF) of at least 0.003 to 0.04 Hz among the autonomic nerve components of the user, based on said heart rate signal measured by said heart rate signal measuring means.
and a biometric signal detecting means for detecting the biometric signals of said user. and means for determining each sleep stage of the user during sleep based on the biometric signals detected by said biometric signal detecting means, and determining the time of each sleep stage.
And a means for calculating a sleep evaluation index for model building based on the time of each sleep stage determined by said means for determining the sleep stage, and and means for constructing a sleep evaluation prediction model equation specific to the user, based on said sleep evaluation index for model construction calculated by said means for calculating a sleep evaluation index and the energy value of said very low frequency component (VLF) at least before bed-entry calculated by said means for calculating an autonomic component; and
and Calculating a predicted sleep evaluation index based on said sleep evaluation prediction model equation constructed by said means of constructing the sleep evaluation prediction model equation and the energy value of at least said extremely low frequency component (VLF) calculated by said means of calculating the autonomic nervous system thereafter.
Based on the calculated predictive sleep evaluation index, the sleep improvement device is equipped with a means for proposing a sleep improvement plan through lifestyle improvement that at least raises the energy value of the extremely low frequency component (VLF) above a certain value.

2. In a sleep improvement method that contributes to the improvement of sleep conditions by providing a sleep improvement plan through lifestyle improvement suited to the user to improve sleep conditions.
and a heart rate signal measuring process for measuring the heart rate signal of the user by the prescribed heart rate signal measuring means, and wherein the processor performing signal processing calculates, based on the heart rate signal measured in the heart rate signal measuring process, an energy value of an extremely low frequency component (VLF) of at least 0.003 to 0.04 Hz of the autonomic nervous system component of the user.
and a biometric signal detection process for detecting biometric signals of the user by the prescribed biometric signal detection means, and a sleep stage determination process in which the processor determines each sleep stage of the user during sleep based on the biometric signals detected in the biometric signal detection process, and determines the time of each sleep stage.
and The processor calculates a sleep evaluation index for model building based on the time of each sleep stage determined in the sleep stage determination process.
and The processor calculates the sleep evaluation index for model building calculated in the sleep evaluation index calculation process and the sleep evaluation index for model building calculated in the sleep evaluation index calculation process and the sleep evaluation index for model building calculated in the sleep evaluation index calculation process.
and based on the energy value of the very low frequency component (VLF) calculated in the autonomic component calculation process at least prior to bed entry. and a process for constructing a sleep evaluation prediction model equation specific to said user, and
and The processor is based on the sleep evaluation prediction model equation constructed in the sleep evaluation prediction model equation construction process and at least the energy value of the very low frequency component (VLF) calculated subsequently in the autonomic calculation process.
and Calculate the predicted sleep rating index at that time. Based on the calculated predictive sleep evaluation index, a sleep improvement plan is proposed through lifestyle improvement such that at least the energy value of the very low frequency component (VLF) is set to a certain value or more. This sleep improvement plan has suggestion steps. A sleep improvement method characterized by the above.
